(19) United States
(12) Patent Application Publication
(10) Pub. No.: US 2012/0059263 A1
(43) Pub. Date: Mar. 8, 2012

(54) PROVIDING A COLOR DOPPLER MODE IMAGE IN AN ULTRASOUND SYSTEM

(75) Inventors: Jae Keun LEE, Seoul (KR); Hyeong Do Lee, Seoul (KR); Yong Ho Lee, Seoul (KR)

(73) Assignee: MEDISON CO., LTD.

(21) Appl. No.: 13/209,805

(22) Filed: Aug. 15, 2011

(30) Foreign Application Priority Data
Sep. 2, 2010 (KR) 10-2010-0085969

(51) Int. Cl. A61B 8/13

(52) U.S. Cl. 600/441

(57) ABSTRACT

Embodiments for providing a color Doppler mode image are disclosed. In one embodiment, by way of non-limiting example, an ultrasound system comprises: an ultrasound data acquisition unit configured to transmit and receive ultrasound signals to and from a living body to acquire first ultrasound data and second ultrasound data; a user input unit configured to receive input information corresponding to a region of interest and a balance threshold value having a predetermined brightness value; and a processing unit configured to form a brightness mode image and a color Doppler mode image corresponding to the region of interest based on the first and second ultrasound data, respectively, form a balance mask for performing a balance process upon the color Doppler mode image based on the brightness mode image and the input information, and perform the balance process upon the color Doppler mode image based on the balance mask.

### CROSS-REFERENCE TO RELATED APPLICATION

The present application claims priority from Korean Patent Application No. 10-2010-0085969 filed on Sep. 2, 2010, the entire subject matter of which is incorporated herein by reference.

### TECHNICAL FIELD

The present disclosure generally relates to ultrasound systems, and more particularly to providing a color Doppler mode image in an ultrasound system.

### BACKGROUND

An ultrasound system has become an important and popular diagnostic tool since it has a wide range of applications. Specifically, due to its non-invasive and non-destructive nature, the ultrasound system has been extensively used in the medical profession. Modern high-performance ultrasound systems and techniques are commonly used to produce two-dimensional or three-dimensional ultrasound images of internal features of an object (e.g., human organs).

The ultrasound system may provide ultrasound images of various modes including a brightness mode (B mode) image representing reflection coefficients of the ultrasound signals reflected from a target object of a living body with a two-dimensional image, a Doppler mode image representing speed of a moving object with spectral Doppler by using a Doppler effect, a color Doppler mode image representing speed of a moving object with colors by using the Doppler effect, and an elastic mode image representing mechanical characteristics of tissues before and after applying a pressure thereto. Particularly, the ultrasound system may transmit and receive ultrasound signals to and from the target object to thereby form Doppler signals corresponding to a region of interest (ROI), which is set on a B mode image. The ultrasound system may further form a color Doppler mode image that represents the speed of the moving object with colors based on the Doppler signals.

The ultrasound system may perform a balance process to eliminate the color Doppler mode image corresponding to regions wherein a brightness value corresponding to a pixel of a brightness mode image is larger than a predetermined balance threshold value upon the color Doppler mode image. This is to remove color artifacts, which may occur by motion of regions such as blood-vessel walls, except interior spaces of the blood vessels, i.e., a lumen in which blood flows. However, this can be a problem since the conventional balance process cannot remove the color artifacts perfectly and decreases quality of the color Doppler mode image.

### SUMMARY

Embodiments for providing a color Doppler mode image in an ultrasound system are disclosed herein. In one embodiment, by way of non-limiting example, an ultrasound system comprises: an ultrasound data acquisition unit configured to transmit and receive ultrasound signals to and from a living body to acquire first ultrasound data and second ultrasound data; a user input unit configured to receive input information corresponding to a region of interest and a balance threshold value having a predetermined brightness value; and a processing unit in communication with the ultrasound data acquisition unit and the user input unit, the processing unit being configured to form a brightness mode image and a color Doppler mode image corresponding to the region of interest based on the first and second ultrasound data, respectively, form a balance mask for performing a balance process upon the color Doppler mode image based on the brightness mode image and the input information, and perform the balance process upon the color Doppler mode image based on the balance mask.

In another embodiment, there is provided a method of providing a color Doppler mode image, comprising: a) forming a brightness mode image based on first ultrasound data for a living body; b) receiving input information corresponding to a region of interest and a balance threshold value having a predetermined brightness value from a user; c) forming a color Doppler mode image corresponding to the region of interest based on second ultrasound data for the living body; d) forming a balance mask for performing a balance process upon the color Doppler mode image based on the brightness mode image and the input information; and e) performing the balance process upon the color Doppler mode image based on the balance mask.

In yet another embodiment, there is provided a computer readable medium comprising computer executable instructions configured to perform the following acts: a) forming a brightness mode image based on first ultrasound data for a living body; b) receiving input information corresponding to a region of interest and a balance threshold value having a predetermined brightness value from a user; c) forming a color Doppler mode image corresponding to the region of interest based on second ultrasound data for the living body; d) forming a balance mask for performing a balance process upon the color Doppler mode image based on the brightness mode image and the input information; and e) performing the balance process upon the color Doppler mode image based on the balance mask.

This summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 is a block diagram showing an illustrative embodiment of an ultrasound system.

FIG. 2 is a schematic diagram showing an example of a brightness mode image and a region of interest.

FIG. 3 is a block diagram showing an illustrative embodiment of an ultrasound data acquisition unit.

FIG. 4 is a flow chart showing a process of performing a balance process upon a color Doppler mode image.

FIG. 5 is a flow chart showing a process of forming a balance mask.

FIG. 6 is a schematic diagram showing an example of an image parameter.

FIG. 7 is a schematic diagram showing another example of the image parameter.

FIG. 8 is a schematic diagram showing yet another example of the image parameter.

FIG. 9 is a schematic diagram showing still yet another example of the image parameter.

### DETAILED DESCRIPTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

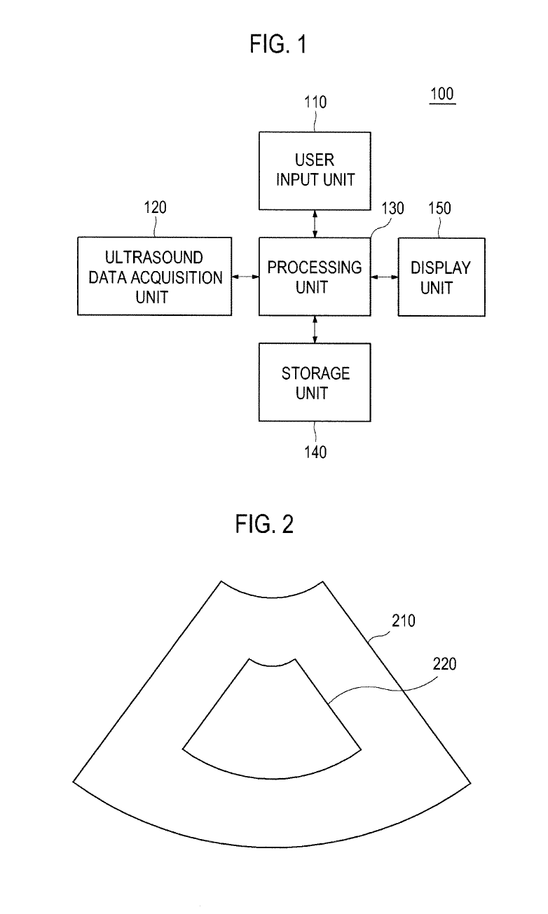

Referring to FIG. 1, an ultrasound system 100 in accordance with an illustrative embodiment is shown. As depicted therein, the ultrasound system 100 may include a user input unit 110. The user input unit 110 may be configured to receive input information for a user. In one embodiment, the input information may include first input information for setting a region of interest (ROI) 220 on a brightness mode image 210 as shown in FIG. 2. The ROI 220 may include a color box for obtaining a color Doppler mode image. The input information may further include second input information for setting a threshold value having a predetermined brightness value (“balance threshold value”) for performing a balance processing upon the color Doppler mode image corresponding to the ROI 220. However, it should be noted herein that the input information may not be limited thereto. The user input unit 110 may include a control panel, a track ball, a mouse, a keyboard and the like.

The ultrasound system 100 may further include an ultrasound data acquisition unit 120. The ultrasound data acquisition unit 120 may be configured to transmit and receive ultrasound signals to and from a living body and output ultrasound data. The living body may include a plurality of target objects (e.g., blood vessels, heart, etc.).

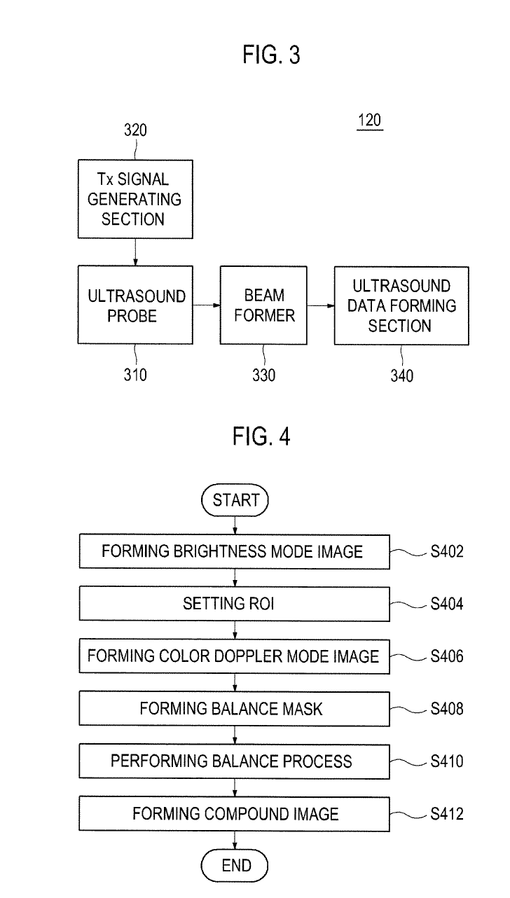

FIG. 3 is a block diagram showing an illustrative embodiment of the ultrasound data acquisition unit 120. Referring to FIG. 3, the ultrasound data acquisition unit 120 may include an ultrasound probe 310. The ultrasound probe 310 may include a plurality of elements (not shown) for reciprocally converting between ultrasound signals and electrical signals. The ultrasound probe 310 may be configured to transmit ultrasound signals to the living body. The ultrasound probe 310 may be further configured to receive ultrasound signals (i.e., ultrasound echo signals) from the living body to output received signals. The received signals may be analog signals. The ultrasound probe 310 may include a convex probe, a linear probe and the like.

The ultrasound data acquisition unit 120 may further include a transmit (Tx) signal generating section 320. The Tx signal generating section 320 may be configured to control the transmission of the ultrasound signals. The Tx signal generating section 320 may be further configured to generate electrical signals (“Tx signals”) for obtaining at least one ultrasound image in consideration of the elements and focal points.

In one embodiment, the Tx signal generating section 320 may be configured to generate first Tx signals for obtaining the brightness mode image. Thus, the ultrasound probe 310 may be configured to convert the first Tx signals provided from the Tx signal generating section 320 into the ultrasound signals, transmit the ultrasound signals to the living body and receive the ultrasound echo signals from the living body to thereby output first received signals. The Tx signal generating section 320 may be further configured to generate second Tx signals for obtaining the color Doppler mode image based on a predetermined ensemble number. The ensemble number may represent the number of transmitting and receiving ultrasound signals in order to obtain Doppler signals corresponding to each of the scan-lines. Thus, the ultrasound probe 310 may be configured to convert the second Tx signals provided from the Tx signal generating section 320 into the ultrasound signals, transmit the ultrasound signals to the living body and receive the ultrasound echo signals from the living body to thereby output second received signals.

The ultrasound data acquisition unit 120 may further include a beam former 330. The beam former 330 may be configured to convert the received signals provided from the ultrasound probe 310 into digital signals. The beam former 330 may be further configured to apply delays to the digital signals in consideration of the elements and the focal points to thereby output digital receive-focused signals.

In one embodiment, the beam former 330 may be configured to convert the first received signals provided the ultrasound probe 310 into first digital signals. The beam former 330 may be further configured to apply delays to the first digital signals in consideration of the elements and the focal points to output first digital receive-focused signals. The beam former 330 may also be configured to convert the second received signals provided from the ultrasound probe 310 into second digital signals. The beam former 330 may be further configured to apply delays to the second digital signals in consideration of the elements and the focal points to output second digital receive-focused signals.

The ultrasound data acquisition unit 120 may further include an ultrasound data forming section 340. The ultrasound data forming section 340 may be configured to form ultrasound data based on the digital receive-focused signals provided from the beam former 330. The ultrasound data forming section 340 may be further configured to perform signal processing (e.g., gain control, etc.) upon the digital receive-focused signals.

In one embodiment, the ultrasound data forming section 340 may be configured to form first ultrasound data based on the first digital receive-focused signals provided from the beam former 330. The first ultrasound data may include radio frequency data. However, it should be noted herein that the first ultrasound data may not be limited thereto. The ultrasound data forming section 340 may be further configured to form second ultrasound data based on the second digital receive-focused signals provided from the beam former 330. The second ultrasound data may be in-phase/quadrature (IQ) data. However, it should be noted herein that the second ultrasound data may not be limited thereto.

Although it is described above that the ultrasound data acquisition unit 120 may be configured to acquire the ultrasound data by transmitting and receiving the ultrasound signals to and from the living body, the ultrasound data acquisition unit 120 may be further configured to acquire the ultrasound data from an external or internal storage unit (not shown) connected to the ultrasound system 100.

Referring back to FIG. 1, the ultrasound system 100 may further include a processing unit 130 in communication with the user input unit 110 and the ultrasound data acquisition unit 120. The processing unit 130 may include a central processing unit, a microprocessor or a graphic processing unit. However, it should be noted herein that the processing unit 130 may not be limited thereto.

FIG. 4 is a flow chart showing a process of performing the balance process upon the color Doppler mode image. The processing unit 130 may be configured to form the brightness mode image based on the first ultrasound data provided from the ultrasound data acquisition unit 120 at step S402 in FIG. 4. The brightness mode image may be displayed on a display unit 150. Thus, the user may set the ROI on the brightness mode image displayed on the display unit 150 by using the user input unit 110.

The processing unit 130 may be configured to set the ROI on the brightness mode image based on the input information (i.e., first input information) provided from the user input unit 110 at step S404 in FIG. 4. Thus, the ultrasound data acquisition unit 120 may transmit and receive the ultrasound data to and from the living body to thereby acquire the second ultrasound data corresponding to the ROI.

The processing unit 130 may be configured to form the color Doppler mode image based on the second ultrasound data provided from the ultrasound data acquisition unit 120 at step S406 in FIG. 4. The methods of the color Doppler mode image are well known in the art. Thus, they have not been described in detail so as not to unnecessarily obscure the present invention.

The processing unit 130 may be configured to form a mask (“balance mask”) for performing the balance process upon the color Doppler mode image based on the input information (i.e., second input information) provided from the user input unit 110 and the brightness mode image at step S408 in FIG. 4.

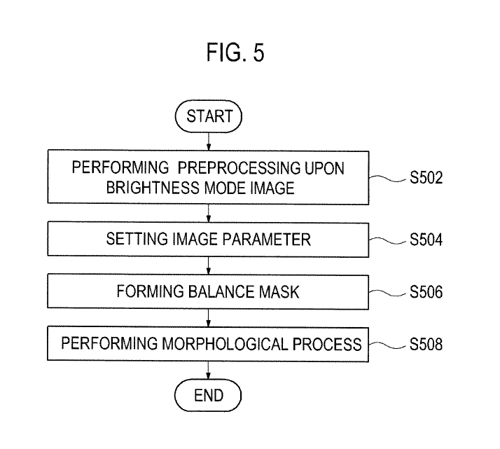

FIG. 5 is a flow chart showing a process of forming the balance mask. The processing unit 130 may be configured to perform a preprocessing upon the brightness mode image to eliminate unnecessary image data at step S502 in FIG. 5. The preprocessing may include a noise elimination process by using an average filter. However, it should be noted herein that the preprocessing may not be limited thereto.

The processing unit 130 may be configured to set an image parameter based on the input information (i.e., second input information) provided from the user input unit 110 and the brightness mode image at step S504 in FIG. 5. The image parameter is a parameter for forming the balance mask. That is, the image parameter is a parameter for securing connectivity of regions (e.g., blood vessels, tissues, etc.) except the inside space of the blood vessels, i.e., a lumen in which the blood flows.

In one embodiment, the processing unit 130 may be configured to calculate a mean brightness value (“global mean brightness value”) of pixels corresponding to the ROI of the brightness mode image based on brightness values corresponding to the pixels.

The processing unit 130 may be further configured to calculate a pivot point value of a contrast stretching curve for controlling brightness levels based on the second input information (i.e., balance threshold value) provided from the user input unit 110 and the global mean brightness value. The processing unit 130 may calculate the pivot point value using equation 1 provided below.

PPV=(V_{GM}+BTV)/2×V_{PPT} (1)

In the equation, PPV represents the pivot point value, V_{GM} represents the global mean brightness value, BTV represents the balance threshold value and V_{PPT} represents a pivot point threshold value. The pivot point threshold value is a predetermined value in consideration of an application and type of the target object. For example, the pivot point threshold value is 0.8 to 1.2.

The processing unit 130 may be configured to calculate a gradient (“curvature”) of the contrast stretching curve based on the second input information (i.e., balance threshold value) and the global mean brightness value. For example, the processing unit 130 may detect a maximum brightness value from the brightness mode image. The processing unit 130 may further calculate a difference between a predetermined brightness value and the maximum brightness value. The processing unit 130 may further set the difference as the gradient of the contrast stretching curve. The methods of calculating the gradient are well known in the art. For example, the processing unit 130 may calculate the gradient using equation 2 provided below.

w=α·x^{d} (2)

In the equation, w represents the gradient, α represents a weight value, which is a predetermined value, x represents a constant value, and d represent a different value between the mean brightness value (i.e., global mean brightness value or local mean brightness value) and the balance threshold value.

As the difference between the global mean brightness value and the balance threshold value gets larger, the gradient becomes larger. As the difference between the global mean brightness value and the balance threshold value gets smaller, the gradient becomes smaller. However, it should be noted herein that the sign may not be limited thereto.

The processing unit 130 may be configured to set a sign of the contrast stretching curve based on the second input information (i.e., balance threshold value) and the pivot point value. The sign may include “+” and “−.” However, it should be noted herein that the sign may not be limited thereto. For example, the processing unit 130 may compare the balance threshold value with the pivot point value. If the balance value is equal to or larger than the pivot point value, then the processing unit 130 may set the sign as “+.” If the balance threshold value is smaller than the pivot point value, then the processing unit 130 may set the sign as “−.”

The processing unit 130 may be configured to set the image parameter based on the second input information, the global mean brightness value, the pivot point value, the gradient and the sign.

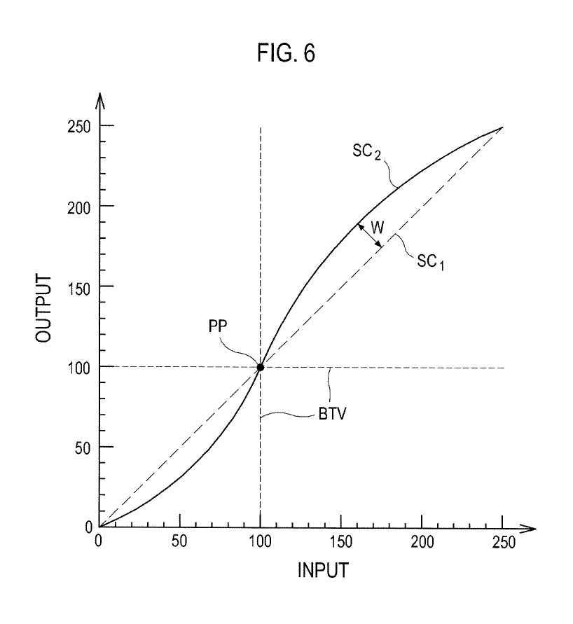

As one example, when the balance threshold value is 100, the global mean brightness value is 100 and the pivot point threshold value is 1.0, the processing unit 130 may set a first contrast stretching curve SC₁ based on the balance threshold value BTV as shown in FIG. 6. The processing unit 130 may further calculate the pivot point value (“100”) by applying the balance threshold value, the global mean brightness value and the pivot threshold value to equation 1. The processing unit 130 may further calculate a difference between the balance brightness value and the global mean brightness value. The processing unit 130 may further calculate the gradient W of the first contrast stretching curve SC₁ based on the calculated difference. The processing unit 130 may further compare the balance threshold value with the pivot point value, and determine that the balance threshold value is equal to the pivot point value. The processing unit 130 may further set the sign of the first contrast stretching curve SC₁ as “+” based on the determining result. The processing unit 130 may further set a second contrast stretching curve SC₂ for setting the brightness values of regions corresponding to pixels that the brightness value is equal to or larger than 100 as a predetermined value (e.g., 255) based on the balance threshold value, the global mean brightness value, the pivot point value, the gradient and the sign, as shown in FIG. 6. The processing unit 130 may further set the second contrast stretching curve SC₂ as the image parameter for forming the balance mask. In FIG. 6, the reference numeral PP represents a pivot point corresponding to the pivot point value. The image parameter shown in FIG. 6 may be used to form the second contrast stretching curve SC₂, which controls the brightness values of the brightness mode image. That is, the image parameter may be used to decrease the brightness values under the pivot point value and increase the brightness values above the pivot point value.

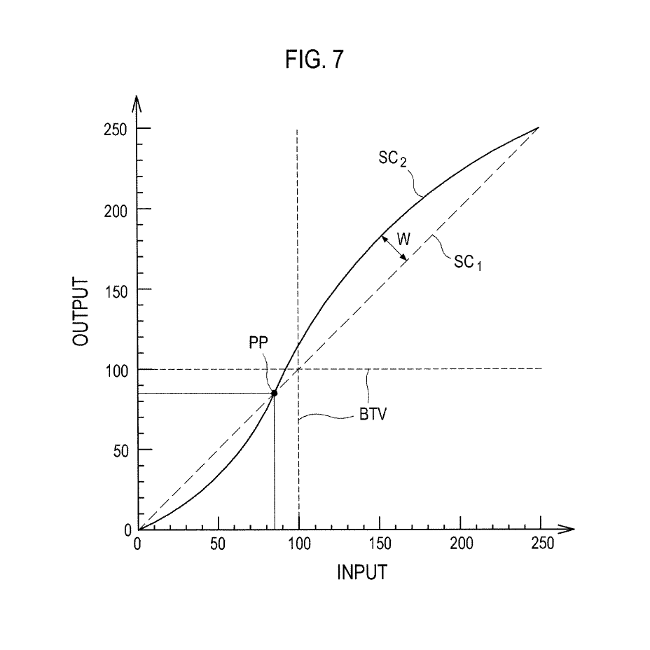

As another example, when the balance threshold value is 100, the global mean brightness value is 100 and the pivot point threshold value is 0.85, the processing unit 130 may set the first contrast stretching curve SC₁ based on the balance threshold value BTV as shown in FIG. 7. The processing unit 130 may further calculate the pivot point value (i.e., 85) by applying the balance threshold value (i.e., 100), the global mean brightness value (i.e., 100) and the pivot point threshold value (i.e., 0.85) to equation 1. The processing unit 130 may further calculate a difference between the balance threshold value and the global mean brightness value. The processing unit 130 may further calculate the gradient W of the first contrast stretching curve SC₁ based on the calculated difference as shown in FIG. 7. The processing unit 130 may further compare the balance threshold value with the pivot point value, and determine that the balance threshold value is larger than the pivot point value. The processing unit 130 may further set the sign of the first contrast stretching curve SC₁ as “+” based on the determining result. The processing unit 130 may further set the second contrast stretching curve SC₂ for setting the brightness values of regions corresponding to pixels that the brightness value is equal to or larger than 85 as a predetermined value (e.g., 255) based on the balance threshold value, the global mean brightness value, the pivot point value, the gradient and the sign, as shown in FIG. 7. The processing unit 130 may further set the second contrast stretching curve SC₂ as the image parameter for forming the balance mask. In FIG. 7, the reference numeral PP represents the pivot point corresponding to the pivot point value.

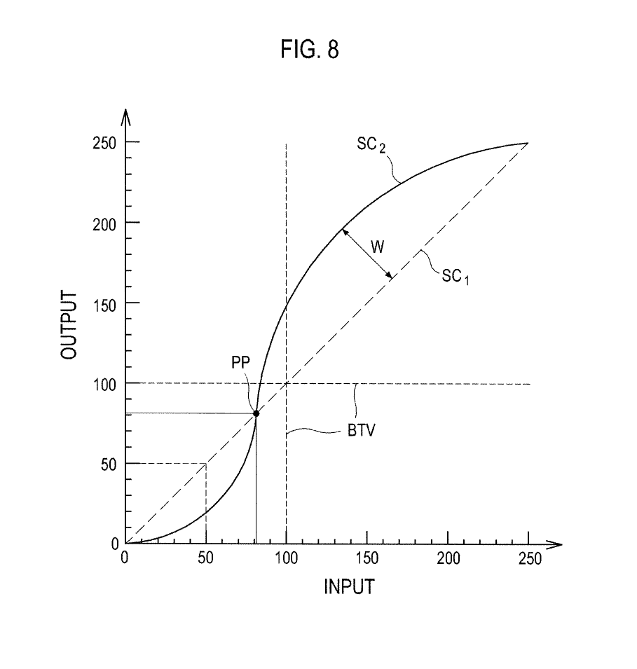

As yet another example, when the balance threshold value is 100, the global mean bright value is 50 and the pivot threshold value is 1.1, the processing unit 130 may set the first contrast stretching curve SC₁ based on the balance threshold value BTV as shown in FIG. 8. The processing unit 130 may further calculate the pivot point value (i.e., 82.5) by applying the balance threshold value (i.e., 100), the global mean brightness value (i.e., 50) and the pivot point threshold value (i.e., 1.1) to equation 1. The processing unit 130 may further calculate a difference between the balance brightness value and the global mean brightness value. The processing unit 130 may further calculate the gradient W of the first contrast stretching curve SC₁ based on the calculated difference as shown in FIG. 8. The processing unit 130 may also compare the balance threshold value with the pivot point value, and determine that the balance threshold value is larger than the pivot point value. The processing unit 130 may further set the sign of the first contrast stretching curve SC₁ as “+” based on the determining result. The processing unit 130 may additionally set the second contrast stretching curve SC₂ for setting the brightness values of regions corresponding to pixels wherein the brightness value is equal to or larger than 82.5 as a predetermined value (e.g., 255) based on the balance threshold value, the global mean brightness value, the pivot point value, the gradient and the sign, as shown in FIG. 8. The processing unit 130 may further set the second contrast stretching curve SC₂ as the image parameter for forming the balance mask. In FIG. 8, the reference numeral PP represents the pivot point corresponding to the pivot point value.

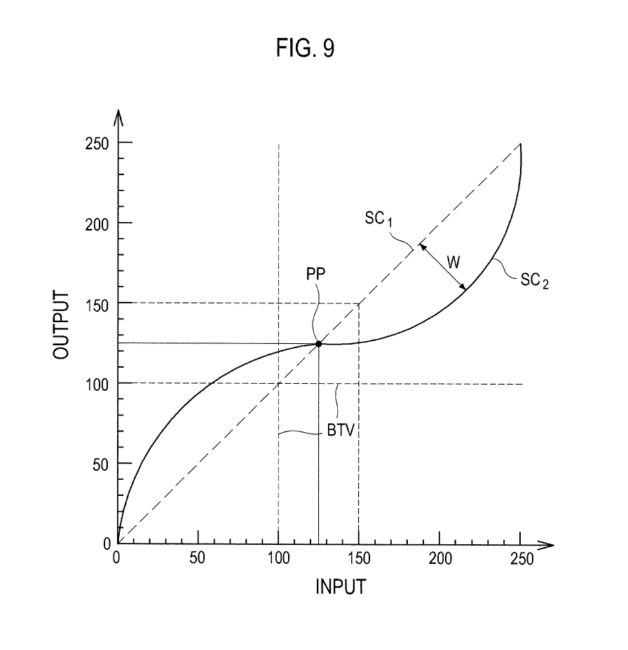

As yet another example, when the balance threshold value is 100, the global mean brightness value is 150 and the pivot point threshold value is 1.0, the processing unit 130 may set the first contrast stretching curve SC₁ based on the balance threshold value BTV as shown in FIG. 9. The processing unit 130 may further calculate the pivot point value (i.e., 125) by applying the balance threshold value (i.e. 100), the global mean brightness value (i.e., 150) and the pivot point threshold value (i.e., 1.0) to equation 1. The processing unit 130 may also calculate a difference between the balance threshold value and the global mean brightness value. The processing unit 130 may further calculate the gradient W of the first contrast stretching curve SC₁ based on the calculated difference as shown in FIG. 9. The processing unit 130 may further compare the balance threshold value with the pivot point value, and determine that the balance threshold value is smaller than the pivot point value. The processing unit 130 may set the sign of the first contrast stretching curve SC₁ as “−” based on the determining result. The processing unit 130 may also set the second contrast stretching curve SC₂ for setting the brightness values of regions corresponding to pixels wherein the brightness value is equal to or larger than 125 as a predetermined value (e.g., 255) based on the balance threshold value, the global mean brightness value, the pivot point value, the gradient and the sign, as shown in FIG. 9. The processing unit 130 may further set the second contrast stretching curve SC₂ as the image parameter for forming the balance mask. In FIG. 9, the reference numeral PP represents the pivot point corresponding to the pivot point value.

In another embodiment, the processing unit 130 may be configured to calculate the global mean brightness value of pixels corresponding to the ROI of the brightness mode image based on the brightness values corresponding to the pixels. The processing unit 130 may be further configured to divide the ROI into a plurality of regions. Each of the regions has at least one pixel. The processing unit 130 may be further configured to calculate a mean brightness value (“local mean brightness value”) of the at least one pixel corresponding to each of the regions. The processing unit 130 may be further configured to calculate a mean brightness value of the global mean brightness value and the local mean brightness value. The processing unit 130 may be also configured to set the image parameter based on the mean brightness value and the second input information provided from the user input unit 110, as mentioned above.

In yet another embodiment, the processing unit 130 may be configured to divide the ROI into a plurality of regions. Each of the regions has at least one pixel. The processing unit 130 may be further configured to calculate the local mean brightness value of the at least one pixel corresponding to each of the regions based on the brightness value of the at least one pixel. The processing unit 130 may be further configured to calculate the pivot point value based on the local mean brightness value and the second input information (i.e., balance threshold value) provided from the user input unit 110. The processing unit 130 may calculate the pivot point value as equation 3 provided below.

PPV=(V_{LM}+BTV)/2×V_{PPT} (3)

In the equation, PPV represents the pivot point value, V_{LM} represents the local mean brightness value, BTV represents the balance threshold value and V_{PPT} represents the pivot point threshold value.

The processing unit 130 may be configured to calculate the gradient of the contrast stretching curve based on the second input information (i.e., balance threshold value) and the local mean brightness value for each of the regions, as mentioned above. The processing unit 130 may be further configured to set the sign of the contrast stretching curve based on the second input information (i.e., balance brightness value) and the pivot point value, as mentioned above. The processing unit 130 may be also configured to set the image parameter based on the second input information, the local mean brightness value, the pivot point value, the gradient and the sign, as mentioned above.

Referring back to FIG. 5, the processing unit 130 may be configured to form the balance mask based on the image parameter at step S506 in FIG. 5. In one embodiment, the processing unit 130 may adjust the brightness values of the pixels of the brightness mode image 210 based on the image parameter shown in FIGS. 6 to 9. The processing unit 130 may further perform an image process for setting the brightness values of regions corresponding to pixels wherein the brightness value is equal to or larger than the pivot point value as a predetermined value (e.g., 255) upon the brightness mode image 210 to form the balance mask.

The processing unit 130 may be configured to perform a morphological process for filling up empty space or enhancing connectivity upon the balance mask at step S508 in FIG. 5. The morphological process may be carried out by sequentially performing dilation and erosion. That is, the balance mask may be removed as many as the predetermined number of pixels and then contracted (erosion).

Although it is described above that the processing unit 130 may use the dilation and erosion as the morphological process, the processing unit 130 may also use opening and closing as the morphological process.

Referring back to FIG. 4, the processing unit 130 may be configured to perform the balance process upon the color Doppler mode image by using the balance mask at step S410 in FIG. 4.

The processing unit 130 may be configured to compound the brightness mode image and the balance-processed color Doppler mode image to form compound image at step S412 in FIG. 4. The methods of compounding the brightness mode image and the color Doppler mode image are well known in the art. Thus, they have not been described in detail so as not to unnecessarily obscure the present invention.

Referring back to FIG. 1, the ultrasound system 100 may include a storage unit 140. The storage unit 140 may store the input information (i.e., first input information and second input information) received by the user input unit 110. The storage unit 140 may further store the ultrasound data (i.e., first ultrasound data and second ultrasound data) acquired by the ultrasound data acquisition unit 120. The storage unit 140 may also store the brightness mode image formed by the processing unit 130. The storage unit 140 may further store the color Doppler mode image formed by the processing unit 130.

The ultrasound system 100 may include the display unit 150. The display unit 150 may display the brightness mode image formed by the processing unit 130. The display unit 150 may further display the compound image formed by the processing unit 130. The display unit 150 may also display the color Doppler mode image formed by the processing unit 130.

In another embodiment, the present invention may provide a computer readable medium comprising computer executable instructions configured to perform the following acts: a) forming a brightness mode image based on first ultrasound data for a living body; b) receiving input information corresponding to a region of interest and a balance threshold value having a predetermined brightness value from a user; c) forming a color Doppler mode image corresponding to the region of interest based on second ultrasound data for the living body; d) forming a balance mask for performing a balance process upon the color Doppler mode image based on the brightness mode image and the input information; and e) performing the balance process upon the color Doppler mode image based on the balance mask. The computer readable medium may comprise a floppy disk, a hard disk, a memory, a compact disk, a digital video disk, etc.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound system, comprising:
an ultrasound data acquisition unit configured to transmit and receive ultrasound signals to and from a living body to acquire first ultrasound data and second ultrasound data;
a user input unit configured to receive input information corresponding to a region of interest and a balance threshold value having a predetermined brightness value; and
a processing unit in communication with the ultrasound data acquisition unit and the user input unit, the processing unit being configured to form a brightness mode image and a color Doppler mode image corresponding to the region of interest based on the first and second ultrasound data, respectively, form a balance mask for performing a balance process upon the color Doppler mode image based on the brightness mode image and the input information, and perform the balance process upon the color Doppler mode image based on the balance mask.

2. The ultrasound system of claim 1, wherein the input information includes:
first input information for setting the region of interest on the brightness mode image; and
second input information for setting the balance threshold value.

3. The ultrasound system of claim 2, wherein the processing unit is configured to:
form the brightness mode image based on the first ultrasound data;
set the region of interest on the brightness mode image based on the first input information;
form the color Doppler mode image based on the second ultrasound data;
set an image parameter for forming the balance mask based on the second input information and the brightness mode image; and
form the balance mask based on the image parameter.

4. The ultrasound system of claim 3, wherein the processing unit is further configured to perform a preprocessing upon the brightness mode image to eliminate unnecessary image data.

5. The ultrasound system of claim 3, wherein the processing unit is further configured to perform a morphological process upon the balance mask to fill up empty space or enhance connectivity upon the balance mask.

6. The ultrasound system of claim 3, wherein the processing unit is configured to:
calculate a mean brightness value of pixels corresponding to the region of interest of the brightness mode image;
calculate a pivot point value of a contrast stretching curve and a gradient of the contrast stretching curve based on the mean brightness value and the balance threshold value, wherein the contrast stretching curve controls brightness levels of the brightness mode image;
set a sign of the contrast stretching curve based on the balance threshold value and the pivot point value; and
set the image parameter based on the mean brightness value, the balance threshold value, the pivot point value, the gradient and the sign.

7. The ultrasound system of claim 6, wherein the processing unit is configured to calculate the pivot point value as a following equation:
PPV=(V_{GB}+BTV)/2×V_{PPT}
wherein PPV is the pivot point value, V_{GB} is the mean brightness value, BTV is the balance threshold value, and V_{PPT} is a pivot point threshold value.

8. The ultrasound system of claim 3, wherein the processing unit is configured to:
calculate a first mean brightness value of pixels corresponding to the region of interest of the brightness mode image;
divide the region of interest into a plurality of regions having a predetermined size;
calculate a second mean brightness value of pixels corresponding to each of the regions;
calculate a mean brightness value of the first mean brightness value and the second mean brightness value for each of the regions;
calculate a pivot point value of a contrast stretching curve and a gradient of the contrast stretching curve based on the mean brightness value and the balance threshold value, wherein the contrast stretching curve controls brightness levels of the brightness mode image;
set a sign of the contrast stretching curve based on the balance threshold value and the pivot point value; and
set the image parameter based on the mean brightness value, the balance threshold value, the pivot point value, the gradient and the sign.

9. The ultrasound system of claim 3, wherein the processing unit is configured to:
divide the region of interest into a plurality of regions having a predetermined size;
calculate a mean brightness value of pixels corresponding to each of the regions;
calculate a pivot point value of a contrast stretching curve and a gradient of the contrast stretching curve based on the mean brightness value and the balance threshold value, wherein the contrast stretching curve controls brightness levels of the brightness mode image;
set a sign of the contrast stretching curve based on the balance threshold value and the pivot point value; and
set the image parameter based on the mean brightness value, the balance threshold value, the pivot point value, the gradient and the sign.

10. The ultrasound system of claim 9, wherein the processing unit is configured to calculate the pivot point value as a following equation:
PPV=(V_{LM}+BTV)/2×V_{PPT}
wherein PPV is the pivot point value, V_{LM} is the mean brightness value, BTV represents the balance threshold value and V_{PPT} represents the pivot point threshold value.

11. A method of providing a color Doppler mode image, comprising:
a) forming a brightness mode image based on first ultrasound data for a living body;
b) receiving input information corresponding to a region of interest and a balance threshold value having a predetermined brightness value from a user;
c) forming a color Doppler mode image corresponding to the region of interest based on second ultrasound data for the living body;
d) forming a balance mask for performing a balance process upon the color Doppler mode image based on the brightness mode image and the input information; and
e) performing the balance process upon the color Doppler mode image based on the balance mask.

12. The method of claim 11, wherein the input information includes:
first input information for setting the region of interest on the brightness mode image; and
second input information for setting the balance threshold value.

13. The method of claim 12, wherein the step d) includes:
d1) setting an image parameter for forming the balance mask based on the second input information and the brightness mode image; and
d2) forming the balance mask based on the image parameter.

14. The method of claim 13, wherein the step d) further includes:
performing a preprocessing upon the brightness mode image to eliminate unnecessary image data.

15. The method of claim 13, wherein the step d) includes:
performing a morphological process upon the balance mask to fill up empty space or enhance connectivity upon the balance mask.

16. The method of claim 13, wherein the step d1) includes:
calculating a mean brightness value of pixels corresponding to the region of interest of the brightness mode image;
calculating a pivot point value of a contrast stretching curve and a gradient of the contrast stretching curve based on the mean brightness value and the balance threshold value, wherein the contrast stretching curve controls brightness levels of the brightness mode image;
setting a sign of the contrast stretching curve based on the balance threshold value and the pivot point value; and
setting the image parameter based on the mean brightness value, the balance threshold value, the pivot point value, the gradient and the sign.

17. The method of claim 16, wherein the pivot point value is calculated as a following equation:
PPV=(V_{GB}+BTV)/2×V_{PPT}
wherein PPV is the pivot point value, V_{GB} is the mean brightness value, BTV is the balance threshold value, and V_{PPT} is a pivot point threshold value.

18. The method of claim 13, wherein the step d1) includes:
calculating a first mean brightness value of pixels corresponding to the region of interest of the brightness mode image;
dividing the region of interest into a plurality of regions having a predetermined size;
calculating a second mean brightness value of pixels corresponding to each of the regions;
calculating a mean brightness value of the first mean brightness value and the second mean brightness value for each of the region;
calculating a pivot point value of a contrast stretching curve and a gradient of the contrast stretching curve based on the mean brightness value and the balance threshold value, wherein the contrast stretching curve controls brightness levels of the brightness mode image;
setting a sign of the contrast stretching curve based on the balance threshold value and the pivot point value; and
setting the image parameter based on the mean brightness value, the balance threshold value, the pivot point value, the gradient and the sign.

19. The method of claim 13, wherein the step d1) includes:
dividing the region of interest into a plurality of regions having a predetermined size;
calculating a mean brightness value of pixels corresponding to each of the regions;
calculating a pivot point value of a contrast stretching curve and a gradient of the contrast stretching curve based on the mean brightness value and the balance threshold value, wherein the contrast stretching curve controls brightness levels of the brightness mode image;
setting a sign of the contrast stretching curve based on the balance threshold value and the pivot point value; and
setting the image parameter based on the mean brightness value, the balance threshold value, the pivot point value, the gradient and the sign.

20. The method of the claim 19, wherein the pivot point value is calculated as following equation:
PPV=(V_{LM}+BTV)/2×V_{PPT}
wherein PPV is the pivot point value, V_{LM} is the mean brightness value, BTV represents the balance threshold value and V_{PPT} represents the pivot point threshold value.

21. A computer readable medium comprising computer executable instructions configured to perform following acts:
a) forming a brightness mode image based on first ultrasound data for a living body;
b) receiving input information corresponding to a region of interest and a balance threshold value having a predetermined brightness value from a user;
c) forming a color Doppler mode image corresponding to the region of interest based on second ultrasound data for the living body;
d) forming a balance mask for performing a balance process upon the color Doppler mode image based on the brightness mode image and the input information; and
e) performing the balance process upon the color Doppler mode image based on the balance mask.
